(19) United States
(12) Patent Application Publication
(10) Pub. No.: US 2008/0102069 A1
(43) Pub. Date: May 1, 2008

(54) TUMOR THERAPY WITH A COMBINATION OF ANTI-HER2 ANTIBODIES

(76) Inventors: Thomas Friess, Diessen-Dettenhofen (DE); Max Hasmann, Muenchen (DE); Werner Scheuer, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/854,584

(22) Filed: Sep. 13, 2007

(30) Foreign Application Priority Data
Sep. 15, 2006 (EP) 06019317.4
Mar. 30, 2007 (EP) 07006616.2

(51) Int. Cl. A61K 39/395; A61P 35/00

(52) U.S. Cl. 424/133.1

(57) ABSTRACT

The present invention provides a method for treating HER2 positive cancer or metastasis of HER2 positive cancer in a patient, who does not respond to a monotherapy with trastuzumab or a monotherapy with pertuzumab or to successive monotherapies with trastuzumab and pertuzumab, comprising co-administering to said patient a therapeutically-effective amount of trastuzumab and pertuzumab simultaneously or sequentially. The invention also provides A kit which comprises trastuzumab and pertuzumab and a package insert instructing the user to co-administer trastuzumab and pertuzumab to a patient suffering from HER2 positive cancer who does not respond to a monotherapy with trastuzumab or a monotherapy with pertuzumab.

### PRIORITY TO RELATED APPLICATION(S)

This application claims the benefit of European Patent Application No. 06019317.4, filed Sep. 15, 2006 and European Patent Application No. 07006616.2, filed Mar. 30, 2007, which is hereby incorporated by reference in its entirety.

### FIELD OF THE INVENTION

The present invention is directed to a method for treating HER2 positive cancer or metastasis of HER2 positive cancer in a patient, who does not respond to a monotherapy with trastuzumab or a monotherapy with pertuzumab or to successive monotherapies with trastuzumab and pertuzumab, comprising co-administering to said patient a therapeutically-effective amount of trastuzumab and pertuzumab simultaneously or sequentially. The present invention relates also to a kit which comprises trastuzumab and pertuzumab and a package insert instructing the user to co-administer trastuzumab and pertuzumab to a patient suffering from HER2 positive cancer who does not respond to a monotherapy with trastuzumab or a monotherapy with pertuzumab.

### BACKGROUND OF THE INVENTION

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

The second member of the HER family, HER2, was originally identified as the product of the transforming gene from neuroblastomas of chemically treated rats. The activated form of the neu proto-oncogene results from a point mutation (valine to glutamic acid) in the transmembrane region of the encoded protein.

Amplification of the human homolog of neu is observed in breast and ovarian cancers and correlates with a poor prognosis (Slamon, et al., Science, 235 (1987) 177-182; Slamon, et al., Science, 244 (1989) 707-712; and U.S. Pat. No. 4,968,603). To date, no point mutation analogous to that in the neu proto-oncogene has been reported for human tumors. Over expression of HER2 (frequently but not uniformly due to gene amplification) has also been observed in other carcinomas including carcinomas of the stomach, endometrium, salivary gland, lung, kidney, colon, thyroid, pancreas and bladder. See, among others, King, et al., Science, 229 (1985) 974; Yokota, et al., Lancet: 1 (1986) 765-767; Fukushige, et al., Mol Cell Biol., 6 (1986) 955-958; Guerin, et al, Oncogene Res., 3 (1988) 21-31; Cohen, et al., Oncogene, 4 (1989) 81-88; Yonemura, et al., Cancer Res., 51 (1991) 1034; Borst et al., Gynecol. Oncol., 38 (1990) 364; Weiner, et al., Cancer Res., 50 (1990) 421-425; Kern, et al., Cancer Res., 50 (1990) 5184; Park, et al., Cancer Res., 49 (1989) 6605; Zhau, et al., Mol. Carcinog., 3 (1990) 254-257; Aasland, et al., Br. J. Cancer 57 (1988) 358-363; Williams, et al., Pathobiology 59 (1991) 46-52; and McCann, et al., Cancer, 65 (1990) 88-92. HER2 may be over expressed in prostate cancer (Gu et al., Cancer Lett. 99 (1996) 185-189; Ross, et al., Hum. Pathol. 28 (1997) 827-833; Ross, et al., Cancer 79 (1997) 2162-2170; and Sadasivan et al., J. Urol. 150 (1993) 126-131).

Antibodies directed against the human HER2 protein products have been described. Hudziak, et al., Mol. Cell. Biol. 9 (1989) 1165-1172 describe the generation of a panel of anti-HER2 antibodies which were characterized using the human breast tumor cell line SK-BR-3. This panel of anti-HER2 antibodies includes, inter alia, the 2C4 and 4D5 antibodies, which are directed to different epitopes of the extracellular domain of HER2. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-over expressing breast tumor cell lines to the cytotoxic effects of TNF-alpha (U.S. Pat. No. 5,677,171). The HER2 antibodies discussed in Hudziak et al. are further characterized in e.g. Fendly, et al. Cancer Research 50 (1990) 1550-1558.

According to WO 01/00245, both the monotherapies of pertuzumab or trastuzumab and the combination of pertuzumab and trastuzumab in treating lung cancer cells in xenograft models revealed effects on tumor growth inhibition. Walshe, J. M., et al., Clin. Breast Cancer 6 (2006) 535-539 relates to the design of a future clinical trial using trastuzumab and pertuzumab in the treatment of HER2 positive breast cancers. Nahta, R. et al, Cancer Research 64 (2004) 2343-2346 describes the combination effects of pertuzumab and trastuzumab in the HER-2-over expressing BT474 breast cancer cell line.

Clinical oncologists are in agreement that the failure of cancer treatment is not necessarily caused by the growth of the primary tumor, which is generally dealt with using surgery, but rather by the metastatic spread into different organs. The regression of primary tumors by different cytotoxic drugs is not always indicative for anti-metastatic activity per se. On the contrary, enhanced metastasis has been observed in response to several anti-cancer drugs (Geldof, et al. Anticancer Res 8 (1988) 1335-40, Murphy, J. Clin. Oncol 11 (1993) 199-201, and De Larco, et al., Cancer Res. 61 (2001) 2857-61). Clearly there exists a need to develop treatment therapies that target not only the primary tumor, but also suppress metastasis. These anti-metastatic activities can e.g. be evaluated by the method according to Schneider, T. et al, Clin. Exp. Metas. 19 (2002) 571-582.

### SUMMARY OF THE INVENTION

The present invention relates to a method for treating HER2 positive cancer or metastasis of HER2 positive cancer in a patient, who does not respond to a monotherapy with trastuzumab or a monotherapy with pertuzumab or to successive monotherapies with trastuzumab and pertuzumab, comprising co-administering to said patient a therapeutically-effective amount of trastuzumab and pertuzumab simultaneously or sequentially.

The present invention relates also to a kit which comprises trastuzumab and pertuzumab and a package insert instructing the user to co-administer trastuzumab and pertuzumab to a patient suffering from HER2 positive cancer who does not respond to a monotherapy with trastuzumab or a monotherapy with pertuzumab.

### DESCRIPTION OF THE FIGURES

FIG. 1 Antitumor activity of (a) monotherapies with trastuzumab or pertuzumab and (b) combined trastuzumab and pertuzumab treatment on KPL-4 primary tumor growth. Mean values of tumor volume (mm³) plotted on the y-axis; number of days after injection of tumor cells plotted on the x-axis. (A) Vehicle (circles), (B) monotherapy with trastuzumab at a loading dose of 30 mg/kg and a maintenance dose of 15 mg/kg once weekly (triangles). (C) monotherapy with pertuzumab at a loading dose of 30 mg/kg and a maintenance dose of 15 mg/kg once weekly (squares) and (D) co-administration of trastuzumab and pertuzumab, each given in the same dose regimen and time schedule as the monotherapies (crosses).

FIG. 2 Effect of (B) monotherapy with trastuzumab (until day 44), (C) monotherapy with pertuzumab (until day 44) and (D) co-administration of trastuzumab and pertuzumab (until day 99) on the prevention or reduction of lung metastasis. Median value of human Alu DNA sequence (ng/ml) quantitated from lung tissue using real-time PCR and plotted on the y-axis.

### DETAILED DESCRIPTION OF THE INVENTION

The term “HER2” according to the invention refers to 185 kDa growth factor receptor also referred to as neu and c-erbB-2 (Slamon, et al., Science 235 (1987) 177-182; Swiss-Prot P04626) whose function is related to neoplastic transformation in human breast cancer cells. Over expression of this protein has been identified in 20-30% of breast cancer patients where it correlates with regionally advanced disease, increased probability of tumor recurrence, and reduced patient survival. As many as 30-40% of patients having gastric, endometrial, salivary gland, non-small cell lung, pancreatic, ovarian, peritoneal, prostate, or colorectal cancers may also exhibit over expression of this protein.

The HER receptor will generally comprise an extracellular domain, which may bind an HER ligand; a lipophilic transmembrane domain, a conserved intracellular tyrosine kinase domain, and a carboxyl-terminal signaling domain harboring several tyrosine residues which can be phosphorylated. The extracellular domain of HER2 comprises four domains, Domain I (amino acid residues from about 1-195), Domain II (amino acid residues from about 196-320), Domain III (amino acid residues from about 321 488), and Domain IV (amino acid residues from about 489-632) (residue numbering without signal peptide). See Garrett, et al., Mol. Cell. 11 (2003) 495-505, Cho, et al., Nature 421 (2003) 756-760, Franklin, et al., Cancer Cell 5 (2004) 317-328, or Plowman, et al., Proc. Natl. Acad. Sci. 90 (1993) 1746-1750 and WO 2006/007398.

Trastuzumab (sold under the trade name Herceptin®) is a recombinant humanized anti-HER2 monoclonal antibody used for the treatment of HER2 over-expressed/HER2 gene amplified metastatic breast cancer. Trastuzumab binds specifically to the same epitope of HER2 as the murine anti-HER2 antibody 4D5 described in Hudziak, et al., Mol. Cell. Biol. 9 (1989) 1165-1172. Trastuzumab is a recombinant humanized version of the murine anti-HER2 antibody 4D5, referred to as rhuMAb 4D5 or trastuzumab) and has been clinically active in patients with HER2-over expressing metastatic breast cancers that had received extensive prior anticancer therapy. (Baselga, et al, J. Clin. Oncol. 14 (1996) 737-744). Trastuzumab and its method of preparation are described in U.S. Pat. No. 5,821,337.

Pertuzumab (Omnitarg®) is another recombinant humanized anti-HER2 monoclonal antibody used for the treatment of HER2 positive cancers. Pertuzumab binds specifically to the 2C4 epitope, a different epitope on the extracellular domain of HER2 as trastuzumab. Pertuzumab is the first in a new class of HER dimerisation inhibitors (HDIs). Through its binding to the HER2 extracellular domain, pertuzumab blocks ligand-activated heterodimerisation of HER2 with other HER family members, thereby inhibiting downstream signalling pathways and cellular processes associated with tumour growth and progression (Franklin, M. C., et al. Cancer Cell 5 (2004) 317-328 and Friess, T, et al. Clin Cancer Res 11 (2005) 5300-5309). Pertuzumab is a recombinant humanized version of the murine anti-HER2 antibody 2C4 (referred to as rhuMAb 2C4 or pertuzumab) and it is described together with the respective method of preparation in WO 01/00245 and WO 2006/007398.

The term “epitope” as used within this application denotes a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. Depending on the size of the antigen to which the epitope belongs, more than one epitope per antigen may be available resulting likewise in the possibility of more than one antibody binding site (=epitope) per antigen.

The “epitope 2C4” is the region in the extracellular domain of HER2 to which the antibody 2C4 binds. In order to screen for antibodies which bind to the 2C4 epitope, a routine cross-blocking assay such as that described in “Ed. Harlow and David Lane, Antibodies, A Laboratory Manual, Cold Spring Harbor Laboratory, (1988)”, can be performed. Alternatively, epitope mapping can be performed to assess whether the antibody binds to the 2C4 epitope of HER2 (e.g. any one or more residues in the region from about residue 22 to about residue 584 of HER2, inclusive). Epitope 2C4 comprises residues from domain II in the extracellular domain of HER2. 2C4 and pertuzumab bind to the extracellular domain of HER2 at the junction of domains I, II and III. See also Franklin, et al., Cancer Cell 5 (2004) 317-328.

The “epitope 4D5” is the region in the extracellular domain of HER2 to which the antibody 4D5 (ATCC CRL 10463) and Trastuzumab bind. This epitope is close to the transmembrane domain of HER2, and within Domain IV of HER2. To screen for antibodies which bind to the 4D5 epitope, a routine cross-blocking assay such as that described in “Ed Harlow and David Lane, Antibodies, A Laboratory Manual, Cold Spring Harbor Laboratory, (1988)”, can be performed. Alternatively, epitope mapping can be performed to assess whether the antibody binds to the 4D5 epitope of HER2 (e.g. any one or more residues in the region from about residue 529 to about residue 625 inclusive).

Antibodies can be generated against, e.g., human, mouse, or rat polypeptides. Antibodies, either polyclonal or monoclonal, specifically recognizing the target antigen, are encompassed by the invention. Such antibodies are raised using standard immunological techniques known to a person skilled in the art. Antibodies may be polyclonal or monoclonal or may be produced recombinantly such as for a humanized antibody. Whether an antibody binds to the same epitope as a known therapeutic antibody can easily be determined in a competitive test system.

Possible epitope overlapping of two antibodies binding to the same target antigen can be detected with the help of a competitive test system, for example, an immunoassay. The extent to which the new antibody competes with the known antibody for the binding to an immobilized target antigen is investigated. For this purpose, an appropriately immobilized target antigen is incubated with the known antibody in labeled form and an excess of the antibody in question. By detection of the bound labeling there can easily be ascertained the extent to which the antibody in question can displace the known antibody from the binding site (=epitope). If there is a displacement of more than 10%, preferably of more than 20%, at the same concentration or at higher concentrations, preferably in the case of 10⁵-fold excess of the antibody in question, referred to the known antibody, then an epitope overlapping is present. That means that the antibody in question binds to the same epitope as the known antibody.

The term “target antigen” relates to a biomolecule which is bound by its corresponding therapeutic antibody. By way of example, the target antigen of a therapeutic antibody to HER2 (=ErbB2 or p 185^{neu}), like Herceptin® or Omnitarg®, is HER2. The target antigen of a therapeutic antibody to EGFR, like Erbitux®, is EGFR. The target antigen of a therapeutic antibody to VEGF, like Avastin®, is VEGF. The target antigen may either be a soluble, i.e. secreted or shed, target antigen or a (cell-)membrane bound target antigen.

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, in: Practice and theory of enzyme immunoassays, Burdon, R. H. and v. Knippenberg, P. H. (eds.), Elsevier, Amsterdam (1990) pp. 221-278; and various volumes of Methods in Enzymology, Colowick, S. P. and Caplan, N. O. (eds.), Academic Press, dealing with immunological detection methods, especially volumes 70, 73, 74, 84, 92 and 121.

The term “over expression” of the HER2 receptor protein is intended to indicate an abnormal level of expression of the HER2 receptor protein in a cell from a tumor within a specific tissue or organ of the patient relative to the level of expression in a normal cell from that tissue or organ. Patients having a cancer characterized by over expression of the HER2 receptor can be determined by standard assays known in the art. Preferably over expression is measured in fixed cells of frozen or paraffin-embedded tissue sections using immunohistochemical (IHC) detection. When coupled with histological staining, localization of the targeted protein can be determined and extent of its expression within a tumor can be measured both qualitatively and semi-quantitatively. Such IHC detection assays are known in the art and include the Clinical Trial Assay (CTA), the commercially available LabCorp 4D5 test, and the commercially available DAKO HercepTest® (DAKO, Carpinteria, Calif.). The latter assay uses a specific range of 0 to 3+ cell staining (0 being normal expression, 3+ indicating the strongest positive expression) to identify cancers having over expression of the HER2 protein (see the Herceptin® (trastuzumab) full prescribing information; September 1998; Genentech, Inc., San Francisco, Calif.). Thus, patients having a cancer characterized by over expression of the HER2 protein in the range of 1+, 2+, or 3+, preferably 2+ or 3+, more preferably 3+ would benefit from the methods of therapy of the present invention.

The term “HER2 positive cancer” refers to a cancer disease such as breast cancer, gastric cancer, endometrial cancer, salivary gland cancer, non-small cell lung cancer, pancreatic cancer, ovarian cancer, peritoneal cancer, prostate cancer, or colorectal cancer, which is characterized by an over expression of HER2 protein. Yokoyama et al., British Journal of Cancer (2006), 95: 1504-1513; Santin et al., Women's Oncology Review (2003), 3: 41-42; Matsui et al., International Journal of Oncology (2005), 27: 681-685; Shimizu et al., Clinical Cancer Research (2005), 11: 2735-2746; Chavez-Blanco et al., BMC Cancer (2004), 4; De Maria et al., Cancer Research (2005), 65: 907-912; Chuang et al., Biochemical and Biophysical Research Communications (2003), 307: 653-659; Bianco, A. R., Journal of Chemotherapy (2004), 16(Suppl. 4): 52-54.

The “cancer” may be, for example, lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or urethra, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. In preferred embodiments, the HER2 positive cancer may be breast cancer, lung cancer, colon cancer, or prostate cancer. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. In a preferred embodiment, the cancer to be treated is a HER2 positive cancer. Also in a preferred embodiment, the cancer is a HER2 positive breast cancer. In a preferred embodiment, the cancer, the patient is suffering from, is a non-responsive, progressive HER2 positive breast cancer, which shows no response to a first-line monotherapy with trastuzumab.

The term “metastasis” according to the invention refers to the transmission of cancerous cells from the “primary tumor” to one or more sites elsewhere in a patient causing secondary, metastatic tumors. A tumor formed by cells that have spread is called a “metastatic cancer” or “metastasis”, e.g. a lung metastasis. The metastasis contains cells originating from the primary tumor or the secondary, metastatic tumor, but differs from the primary or secondary, metastatic tumor e.g. by the site of primary or secondary, metastatic tumor. E.g. the site of primary or secondary, metastatic tumor is a breast cancer and the metastasis is a lung metastasis). Means to determine if a cancer has metastasized are known in the art and include tumor marker tests, bone scan, chest X-ray, computed tomography (CT), computerized axial tomography (CAT), molecular resonance imaging (MRI), positron emission tomography (PET), single photon emission computed tomography (SPECT), fluorescence imaging (FI), and bioluminescent imaging (BLI) (see e.g. Helms, M. W, et al., Contributions to microbiology 13 (2006) 209-231 and Pantel, K., et al., JNCI 91 (1999) 1113-1124).

The term “advanced cancer” refers to a stage of cancer in which the disease has spread by metastasis from the primary site (primary tumor) to other parts of the body, directly or by traveling through the network of lymph glands (lymphatics) or in the bloodstream and thus forming metastasis. When the cancer has spread only to the surrounding areas e.g. to nearby tissues or lymph nodes, it is called locally advanced.

The term “breast cancer” refers to the uncontrolled growth of abnormal breast cells. It includes ductal carcinoma in situ, invasive ductal carcinoma, lobular carcinoma in situ, invasive lobular carcinoma, medullary carcinoma, Paget's disease of the nipple and metastatic breast cancer. Preferably the breast cancer is primary tumor after a first-line monotherapy with trastuzumab. Or alternatively the breast cancer is a locally advanced breast cancer, which may be inoperable, and/or the breast cancer is a metastatic breast cancer after a first-line monotherapy with trastuzumab. Preferably such breast cancer, e.g. the primary tumor, does not respond to a first-line, monotherapy with trastuzumab.

According to RECIST criteria tumor response for solid tumors (Therasse et al., J. Nat. Cancer Institute. 92 (2000) 205-216) is categorized in dependency of the volume progression or regression of the tumors (e.g. measured via CT) into four levels: complete response (CR) or partial response (PR), stable disease (SD) and progressive disease (PD) (see Table 1). Furthermore the European Organization for Research and Treatment of Cancer (EORTC) proposed a categorization into four levels in dependency of the metabolism of the tumors measured via 2-[¹⁸F]-Fluoro-2-deoxyglucose positron emission tomography (FDG-PET) (Young, H., et al., Eur J Canc 35 (1999) 1773-1782 and Kellof, G. J., et al, Clin Canc Res 11 (2005) 2785-2808): complete metabolic response (CMR) or partial metabolic response (PMR), stable metabolic disease (SMD) and progressive metabolic disease (PMD) (see Table 2).

TABLE 1
CT-Criteria (ace. to RECIST)

| | |
|---|---|
| CT -measurement: Change in sums | RECIST |
| longest diameters | |
| Disappearance; conformed at 4 weeks | CR |
| (after treatment start) | |
| 30% decrease; confirmed at 4 weeks | PR |
| Neither PR nor PD criteria met | SD |
| 20% increase, no CR, PR, SD documented | PD |
| before increased disease | |

TABLE 2
Proposed FDG-PET criteria
(acc. to EORTC, see Young H., et al., Eur J Canc 35 (1999) 1773-1782)

| | Proposed |
|---|---|
| | FDG-PET |
| PET-measurement | criteria |
| Complete resolution of 2- | CMR |
| [¹⁸F]-Fluoro-2- | |
| deoxyglucose (FDG) | |
| tumour uptake | |
| Reduction of a minimum | PMR |
| of 15-25% of standardized | |
| uptake value (SUV) after | |
| one treatment cycle, and of | |
| >25% after more than one | |
| treatment cycle | |
| Increase of standardized | SMD |
| uptake value (SUV) <25% | |
| or decrease of SUV <15% | |
| No visible increase the | |
| extent of FDG tumour | |
| (>20% of longest | |
| dimension) uptake | |
| Increase of SUV >25% | PMD |
| Visible increase of FDG | |
| tumour uptake (>20% of | |
| longest dimension) | |
| Appearance of new FDG | |
| uptake in metastatic lesions | |

“Non-Response (NR)” and “Response (RE)” according to this invention are established based on data acquired by the combination of computer tomography (CT) and 2-[¹⁸F]-Fluoro-2-deoxyglucose positron emission tomography (FDG-PET) (Kellof, G. J., et al, Clin Canc Res 11 (2005) 2785-2808 and Young H., et al., Eur J Canc 35 (1999) 1773-82) using both the RECIST and FDG-PET criteria described above. Accordingly Non-Response (NR) and Response (RE) according to this invention are determined as follows:

Non-Response (NR): SD or PD is established via CT-RECIST criteria (Table 1) and at the same time SMD or PMD is established via FDG-PET (Table 2). Thus the following four cases for combined CT and PET measurement signify Non-Response (NR): SD and SMD, SD and PMD, PD and SMD, and PD and PMD, preferably SD and PMD, PD and SMD, and PD and PMD, more preferably PD and PMD and still more preferably PD.

Response (RE): includes all other possible results for combined CT and FDG-PET measurement, preferably Response (RE) means one of the following four cases for combined CT and PET measurement: CR and CMR, PR and CMR, CR and PMR, and PR and PMR.

Usually Non-Response (NR) and Response (RE) are determined at around 3 to 8 weeks, preferably at 4 to 6 weeks, after treatment start. This response determination is usually repeated at intervals of 4 to 8 weeks. The treatment with the combination of trastuzumab and pertuzumab is started at earliest after the first determination of Non-Response (NR) or, alternatively of progression, of the patient suffering of the HER2 positive cancer, preferably during or after a first-line monotherapy with trastuzumab.

As used herein, the term “patient” preferably refers to a human in need of treatment to treat cancer, or a precancerous condition or lesion. In the context of the invention the patient is a patient suffering from HER2 positive cancer, who is need of treatment of said HER2 positive cancer or of the metastasis of said HER2 positive cancer.

However, the term “patient” can also refer to non-human animals, preferably mammals such as dogs, cats, horses, cows, pigs, sheep and non-human primates, among others, that are in need of treatment.

The term “group” refers to a group of patients as well as a sub-group of patients.

The term “first-line therapy” as used herein refers to the first type of drug therapy given for the treatment of cancer or metastasis. This can be an adjuvant or neoadjuvant chemotherapy or immunotherapy offered initially following diagnosis and/or surgery. The term “adjuvant chemotherapy or immunotherapy” as used herein refers a treatment after surgery with the intention of prevent cancer from coming back, the term “neoadjuvant chemotherapy or immunotherapy” as used herein refers to a treatment given prior to surgery with the idea of decreasing the tumor size. The term “chemotherapy” as used herein refers to cancer chemotherapy which is the use of chemical or biochemical substances, like cytotoxic drugs such 5-fluoruracil, or targeted therapies with monoclonal antibodies such as trastuzumab, or with kinase inhibitors such as erlotinib, to treat cancer.

The term “monotherapy” as used herein refers to a therapy with a single chemical or biochemical substance compared to the term “combination therapy” which refers to a therapy with two or more chemical or biochemical substances.

The term “first-line monotherapy” as used herein refers to the first-line therapy as defined above with a single chemical or biochemical substance (in contrast to the term “first-line combination therapy” which refers to a first-line therapy with two or more chemical or biochemical substances).

The invention comprises the use of trastuzumab and pertuzumab, either in one single or two formulations, for the manufacture of a medicament for the treatment of HER2 positive cancer or metastasis of HER2 positive cancer in a patient, who does not respond to a monotherapy with trastuzumab or pertuzumab, preferably a first-line monotherapy with trastuzumab, or to successive monotherapies with trastuzumab and pertuzumab, comprising co-administering to said patient trastuzumab and pertuzumab simultaneously or sequentially.

The invention further comprises the use of trastuzumab and pertuzumab for the manufacture of a medicament for the treatment of HER2 positive cancer or metastasis of HER2 positive cancer in a patient, who does not respond to a first-line monotherapy with trastuzumab, comprising co-administering to said patient trastuzumab and pertuzumab simultaneously or sequentially.

The invention further comprises the use of pertuzumab for the manufacture of a medicament for the treatment of HER2 positive cancer or metastasis of HER2 positive cancer in a patient, who does not respond to a first-line monotherapy with trastuzumab, comprising co-administering to said patient trastuzumab and pertuzumab simultaneously or sequentially.

The invention further comprises the use of trastuzumab and pertuzumab for the manufacture of a medicament for the treatment of HER2 positive cancer or metastasis of HER2 positive cancer in a patient, who responds neither to a monotherapy with trastuzumab nor to a monotherapy with pertuzumab, comprising co-administering to said patient trastuzumab and pertuzumab simultaneously or sequentially.

The invention further comprises the use of pertuzumab for the manufacture of a medicament for the treatment of HER2 positive cancer or metastasis of HER2 positive cancer in a patient, who responds neither to a monotherapy with trastuzumab nor to a monotherapy with pertuzumab, comprising co-administering to said patient trastuzumab and pertuzumab simultaneously or sequentially.

The non-response (NR) of said patient, who does not respond to a monotherapy with trastuzumab or pertuzumab, or to successive monotherapies with trastuzumab and pertuzumab is established according to the above definition of NR.

The term “successive monotherapies with trastuzumab and pertuzumab” refers to two separate monotherapies with trastuzumab and pertuzumab, wherein between the last dose of the first monotherapy, e.g. with trastuzumab, and the first dose of the second monotherapy, e.g. with pertuzumab, lays an interval of at least 3 weeks, preferably 4 weeks, and more preferably 5 or 6 weeks.

In a preferred embodiment of the invention the treatment is directed to HER2 positive cancer in a patient, who does not respond to a monotherapy with trastuzumab or pertuzumab, preferably to a first-line monotherapy with trastuzumab, or to successive monotherapies with trastuzumab and pertuzumab, comprising co-administering to said patient trastuzumab and pertuzumab simultaneously or sequentially.

In a preferred embodiment of the invention the treatment is directed to HER2 positive cancer in a patient, who does not respond to a monotherapy with trastuzumab or pertuzumab, preferably to a first-line monotherapy with trastuzumab, comprising co-administering to said patient trastuzumab and pertuzumab simultaneously or sequentially.

In another preferred embodiment of the invention the treatment is directed to the metastasis of HER2 positive cancer in a patient, who does not respond to a monotherapy with trastuzumab or pertuzumab, preferably to a first-line monotherapy with trastuzumab, or to successive monotherapies with trastuzumab and pertuzumab, comprising co-administering to said patient trastuzumab and pertuzumab simultaneously or sequentially.

In another preferred embodiment of the invention the treatment is directed to the metastasis of HER2 positive cancer in a patient, who does not respond to a monotherapy with trastuzumab or pertuzumab, preferably to a first-line monotherapy with trastuzumab, comprising co-administering to said patient trastuzumab and pertuzumab simultaneously or sequentially.

One other aspect of the invention is the use of trastuzumab for the manufacture of a medicament for preventing or reducing metastasis in a patient suffering from HER2 positive cancer, who does not respond to a monotherapy with trastuzumab nor to a monotherapy with pertuzumab, characterized in that trastuzumab and pertuzumab are co-administered simultaneously or sequentially.

Preferably said use is characterized in that the patient does not respond to a first-line monotherapy with trastuzumab.

Preferably said use characterized in that the HER2 positive cancer is a locally advanced cancer and/or a metastatic cancer.

Preferably said use characterized in that the HER2 positive cancer is a locally advanced cancer and/or a metastatic cancer and the patient does not respond to a first-line monotherapy with trastuzumab.

Preferably said use is characterized in that the HER2 positive cancer is breast cancer.

Preferably said use is characterized in that the metastasis is a metastasis of the lung.

Preferably said use is characterized in that the HER2 positive cancer is a locally advanced cancer and/or a metastatic cancer, preferably breast cancer, and the patient does not respond to a first-line monotherapy with trastuzumab, and the metastasis is a metastasis of the lung.

The terms “medicament for preventing metastasis” or “medicament for reducing metastasis” as used herein refer to use of the medicament as a prophylactic agent against metastasis in patient suffering from HER2 positive cancer, does not respond to a monotherapy with trastuzumab or pertuzumab, preferably to a first-line monotherapy with trastuzumab, or to successive monotherapies with trastuzumab and pertuzumab, in this way inhibiting or reducing a further transmission of cancerous cells from the primary or metastatic tumor to one or further sites elsewhere in a patient. This means that the metastasis of the primary tumor or the, metastatic tumor e.g. locally advanced cancer, is prevented, delayed, or inhibited. Preferably the metastasis of the lung is prevented or reduced, which means that metastatic transmission of cancerous cells from the primary tumor to the lung is prevented or reduced. In this context, there can exist different therapies with different anticancer agents, e.g. one therapy aims to treat the primary tumor or a metastatic tumor, such as a locally advanced cancer, and another therapy aims to prevent initial metastasis of a primary tumor or further metastasis of metastatic tumor, such as a locally advanced cancer. E.g. such a prevention treatment can significantly reduce the incidence of a locally advanced breast cancer at other sites of the body, like e.g. the lung, the liver, etc.

In a preferred embodiment of the invention, the patient, suffering from HER2 positive cancer, does not respond to a monotherapy with trastuzumab or pertuzumab, preferably to a first-line monotherapy with trastuzumab.

In another preferred embodiment of the invention, the patient, suffering from HER2 positive cancer, does not respond to successive monotherapies with trastuzumab and pertuzumab.

In another preferred embodiment of the invention, the patient, suffering from HER2 positive cancer, does not respond to a monotherapy with trastuzumab, preferably to a first-line monotherapy with trastuzumab.

In another preferred embodiment of the invention, the patient, suffering from HER2 positive cancer, does not respond to a monotherapy with pertuzumab.

In a preferred embodiment of the invention, trastuzumab and pertuzumab are co-administered to the patient, suffering from HER2 positive cancer.

The terms “use of trastuzumab and pertuzumab for the manufacture of a medicament” or “use of pertuzumab for the manufacture of a medicament” relate to the manufacturing of a medicament for use in the indication as specified herein and in particular for use in the treatment of tumors, tumor metastases, or cancer in general.

The invention further comprises a method of treating HER2 positive cancer or metastasis of HER2 positive cancer in a patient, who does not respond to a monotherapy with trastuzumab or pertuzumab, preferably to a first-line monotherapy with trastuzumab, or to successive monotherapies with trastuzumab and pertuzumab, comprising co-administering to said patient a therapeutically-effective amount of trastuzumab and pertuzumab simultaneously or sequentially.

The invention further comprises a method of treating HER2 positive cancer or metastasis of HER2 positive cancer in a patient, who does not respond to a monotherapy with trastuzumab or pertuzumab, preferably to a first-line monotherapy with trastuzumab, comprising co-administering to said patient a therapeutically-effective amount of trastuzumab and pertuzumab simultaneously or sequentially.

The invention further comprises a method of treating HER2 positive cancer in a patient, who does not respond to a monotherapy with trastuzumab or pertuzumab, preferably to a first-line monotherapy with trastuzumab, comprising co-administering to said patient a therapeutically-effective amount of trastuzumab and pertuzumab simultaneously or sequentially.

The invention further comprises a method of treating metastasis of HER2 positive cancer in a patient, who does not respond to a monotherapy with trastuzumab or pertuzumab, preferably to a first-line monotherapy with trastuzumab, comprising co-administering to said patient a therapeutically-effective amount of trastuzumab and pertuzumab simultaneously or sequentially.

In an embodiment of the present invention, the HER2 positive cancer involved in the above treatment methods is a primary tumor. In another embodiment of the present invention, the HER2 positive cancer is a locally advanced cancer or a metastatic cancer. In yet another embodiment of the present invention, the HER2 positive cancer is breast cancer.

In an embodiment of the present invention, the metastasis of HER2 positive cancer is metastasis of the lung.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed an overall beneficial course of action.

It is self-evident that the antibodies are administered to the patient in therapeutically effective amount which is the amount of the subject compound or combination that will elicit the biological or medical response of a tissue, system, animal or human that is being sought by the researcher, veterinarian, medical doctor or other clinician.

The terms “co-administration of trastuzumab and pertuzumab” or “co-administering trastuzumab and pertuzumab” refer to the administration of trastuzumab and pertuzumab as one single formulation or as two separate formulations (one for trastuzumab and one for pertuzumab). The co-administration can be simultaneous or sequential in either order, wherein there is a time period while both (or all) active agents simultaneously exert their biological activities. As such, the “co-administration” of trastuzumab and pertuzumab refers to a combination therapy (as defined above) involving trastuzumab and pertuzumab. This is as opposed to a monotherapy wherein only one agent exerts its biological activity. In successive monotherapies of trastuzumab and pertuzumab, only one agent is exerting its activity at a time whereas, in a sequential co-administration of trastuzumab and pertuzumab, there is a time period in which both agents are simultaneously exerting their biological activities. As stated previously, successive monotherapies involving two agents involves an interval of at least 3 weeks between the last dose of the monotherapy with the first agent and the first dose of the monotherapy with the second agent. In contrast, as stated below, the sequential co-administration of two agents involves the administration of the second agent within 7 days from the dose of the first agent.

If one single formulation of trastuzumab and pertuzumab is used, trastuzumab and pertuzumab are co-administered simultaneously. If two separate formulations (one for trastuzumab and one for pertuzumab) are used, trastuzumab and pertuzumab are co-administered either simultaneously (e.g. through one single continuous infusion or through two separate continuous infusions at the same time) or sequentially. When both antibodies are co-administered sequentially the dose is administered either on the same day in two separate administrations, e.g. two separate continuous infusions at different times, or one of the antibodies is administered on day 1 and the second antibody is co-administered on day 2 to day 7, preferably on day 2 to 4. Thus the term “sequentially” means within 7 days after the dose of the first antibody, preferably within 4 days after the dose of the first antibody; and the term “simultaneously” means at the same time. The terms “co-administration” with respect to the maintenance doses of trastuzumab and pertuzumab mean that the maintenance doses can be either co-administered simultaneously, e.g. during one continuous infusion, if the treatment cycle is appropriate for both antibodies, e.g. every 3 weeks, or the maintenance doses are co-administered sequentially, either within one or within several days, e.g. the maintenance dose of one of the antibodies is administered approximately every 3 weeks, and the maintenance dose of the second antibodies is co-administered also every 3 weeks. Also other treatment cycles/usually from 1 to 4 weeks, preferably from 2 to 3 weeks, may be used for both antibodies.

The amount of trastuzumab and pertuzumab co-administration and the timing of co-administration will depend on the type (species, gender, age, weight, etc.) and condition of the patient being treated and the severity of the disease or condition being treated. Trastuzumab and pertuzumab are suitably co-administered to the patient at one time or over a series of treatments. Depending on the type and severity of the disease, about 1 μg/kg to 50 mg/kg (e.g. 0.1-20 mg/kg) of trastuzumab or pertuzumab is an initial candidate dosage for co-administration to the patient, whether, for example, by one or more separate administrations, or by continuous infusion. In one embodiment, the initial infusion time for the trastuzumab or pertuzumab may be longer than subsequent infusion times, for instance approximately 90 minutes for the initial infusion, and approximately 30 minutes for subsequent infusions (if the initial infusion is well tolerated). The preferred dosage of trastuzumab or pertuzumab will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be co-administered to the patient. Such doses may be co-administered intermittently, e.g. every week or every three weeks, preferably every 3 weeks, (e.g. such that the patient receives from about two to about twenty, e.g. about six doses of trastuzumab and pertuzumab each). An initial higher loading dose, followed by one or more lower doses may be administered. In one embodiment, trastuzumab or pertuzumab are co-administered as a loading dose of 4 mg/kg as continuous infusion and subsequent 3-weekly infusions of 2 mg/kg to 6 mg/kg, preferably 2 mg/kg, administered as continuous infusion.

In a preferred embodiment, the medicament is useful for preventing or reducing metastasis in such a patient suffering from HER2 positive cancer, increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, the medicament is useful for increasing the response rate in a group of patients.

In a preferred embodiment, the medicament is useful for reducing metastasis a patient suffering from HER2 positive cancer.

In the context of this invention, additional other cytotoxic, chemotherapeutic or anti-cancer agents, or compounds that enhance the effects of such agents may be used in the trastuzumab and pertuzumab combination treatment of HER2 positive cancer or metastasis of HER2 positive cancer in a patient, who does not respond to a monotherapy with trastuzumab or pertuzumab, preferably a first-line monotherapy with trastuzumab, or to successive monotherapies with trastuzumab and pertuzumab. Preferably the trastuzumab and pertuzumab combination treatment is used without such additional cytotoxic, chemotherapeutic or anti-cancer agents, or compounds that enhance the effects of such agents.

Such agents include, for example: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6 MP), 6-thioguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and paclitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ethyol®), dactinomycin, mechlorethamine (nitrogen mustard), streptozocin, cyclophosphamide, lomustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil. Preferably the trastuzumab and pertuzumab combination treatment is used without such additional agents.

In the context of this invention, an anti-hormonal agent may be used in the trastuzumab and pertuzumab combination treatment of HER2 positive cancer or metastasis of HER2 positive cancer in a patient, who does not respond to a monotherapy with trastuzumab or pertuzumab, preferably a first-line monotherapy with trastuzumab, or to successive monotherapies with trastuzumab and pertuzumab. As used herein, the term “anti-hormonal agent” includes natural or synthetic organic or peptidic compounds that act to regulate or inhibit hormone action on tumors. Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (luteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinyl-carbonyl)-D-lysyl-L-leucyl-N-6-(1-methylethyl)-L-lysyl-L-proline (e.g. Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide(2-methyl-N-[4,20-nitro-3-(trifluoromethyl)phenylpropanamide), commercially available as Eulexin® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR (retinoic acid receptor), RXR (retinoid X receptor), TR (thyroid receptor), VDR (vitamin-D receptor), and the like. Preferably the trastuzumab and pertuzumab combination treatment is used without such additional anti-hormonal agent. The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, additional antiproliferative agents may be used in the trastuzumab and pertuzumab combination treatment of HER2 positive cancer or metastasis of HER2 positive cancer in a patient, who does not respond to a monotherapy with trastuzumab or pertuzumab, preferably a first-line monotherapy with trastuzumab, or to successive monotherapies with trastuzumab and pertuzumab, including, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217. Preferably the trastuzumab and pertuzumab combination treatment is used without such additional antiproliferative agents.

In the context of this invention, an effective amount of ionizing radiation may be carried out and/or a radiopharmaceutical may be used in addition to the trastuzumab and pertuzumab combination treatment of HER2 positive cancer or metastasis of HER2 positive cancer in a patient, who does not respond to a monotherapy with trastuzumab or pertuzumab, preferably a first-line monotherapy with trastuzumab, or to successive monotherapies with trastuzumab and pertuzumab. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes. Preferably the trastuzumab and pertuzumab combination treatment is used without such ionizing radiation.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The antibodies are administered to a patient according to known methods, by intravenous administration as a bolus or by continuous infusion over a period of time, by intramuscular, intraperitoneal, intracerobrospinal, subcutaneous, intra-articular, intrasynovial, or intrathecal routes. Intravenous or subcutaneous administration of the antibodies is preferred.

The invention further comprises a kit comprising trastuzumab and pertuzumab, either in the form of one single or two separate formulations, and a package insert instructing the user to administer trastuzumab and pertuzumab to a patient suffering from HER2 positive cancer, who does not respond to a monotherapy with trastuzumab or a monotherapy with pertuzumab. In an embodiment of the present invention, the patient does not respond to a first-line monotherapy with trastuzumab.

The term “package insert” refers to instructions customarily included in commercial packages of therapeutic products, which may include information about the indications, usage, dosage, administration, contraindications and/or warnings concerning the use of such therapeutic products.

In a preferred embodiment, the aforementioned kit may further include a pharmaceutically-acceptable carrier. The kit may further include a sterile diluent.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

### Pharmaceutical Formulations

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Preferred lyophilized HER2 antibody formulations are described in WO 97/04801.

The formulations according to the invention may be two separate formulations for each of the antibodies trastuzumab and pertuzumab. Alternatively the formulation herein may also contain both antibodies trastuzumab and pertuzumab in one formulation.

Additionally, the composition may further comprise a chemotherapeutic agent, cytotoxic agent, cytokine, growth inhibitory agent, anti-hormonal agent, EGFR-targeted drug, anti-angiogenic agent, and/or cardioprotectant. Such molecules are suitably present in combination in amounts that are effective for the purpose intended.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The following examples and figures are provided to aid the understanding of the present invention, the true scope of which is set forth in the appended claims. It is understood that modifications can be made in the procedures set forth without departing from the spirit of the invention.

### EXAMPLE 1

The current study examined the antitumor activity of a) the combination of trastuzumab and pertuzumab and b) the treatment with trastuzumab or pertuzumab alone, in human breast xenograft model. Further aims of the study were to examine the effects of treatment on metastasis.

Test Agents

Trastuzumab was provided as a 25 mg/ml stock solution in Histidine-HCl, alpha-alpha Trehalose (60 mM), 0.01% Polysorb, pH 6.0 (Herceptin®). Pertuzumab was provided as a mg/ml stock solution in Histidine-HCl, sucrose (240 mM), 0.02% Polysorb, pH 6.0 (Omnitarg®). Both solutions were diluted appropriately in PBS for injections.

Cell Lines and Culture Conditions

The human breast cancer cell line KPL-4 has been established from the malignant pleural effusion of a breast cancer patient with an inflammatory skin metastasis and overexpresses ErbB family receptors. (Kurebayashi et al. Br. J. Cancer 79 (1999) 707-17) Tumor cells are routinely cultured in DMEM medium (PAA Laboratories, Austria) supplemented with 10% fetal bovine serum (PAA) and 2 mM L-glutamine (Gibco) at 37° C. in a water-saturated atmosphere at 5% CO₂. Culture passage is performed with trypsin/EDTA 1× (PAA) splitting twice/week. Cell passage P6 was used for in vivo study.

Animals

SCID beige (C.B.-17) mice; age 10-12 weeks; body weight 18-20 g (Charles River, Sulzfeld, Germany) are maintained under specific-pathogen-free condition with daily cycles of 12 h light/12 h darkness according to international guidelines (GV-Solas; Felasa; TierschG). After arrival, animals are housed in the quarantine part of the animal facility for one week to get accustomed to new environment and for observation. Continuous health monitoring is carried out on regular basis. Diet food (Alltromin) and water (acidified pH 2.5-3) are provided ad libitum.

Tumor Growth Inhibition Studies In Vivo

Tumor cells were harvested (trypsin-EDTA) from culture flasks (Greiner TriFlask) and transferred into 50 ml culture medium, washed once and resuspended in PBS. After an additional washing step with PBS and filtration (cell strainer; Falcon 100 μm) the final cell titer was adjusted to 0.75×10⁸/ml. Tumor cell suspension was carefully mixed with transfer pipette to avoid cell aggregation. Anesthesia was performed using a Stephens's inhalation unit for small animals with preincubation chamber (plexiglas), individual mouse nose-mask (silicon) and Isoflurane (Pharmacia-Upjohn, Germany) in a closed circulation system. Two days before injection the fur of the animals was shaved. For intra mammary fat pad (i.m.f.p.) injection, cells were injected orthotopically at a volume of 20 μl into the right penultimate inguinal mammary fat pad of each anesthetized mouse. For the orthotopic implantation, the cell suspension was injected through the skin under the nipple. Tumor cell injection corresponds to day 1 of the experiment.

Monitoring

Animals were controlled daily for detection of clinical symptoms of adverse effects. For monitoring throughout the experiment, the body weight of the animals was documented two times weekly and the tumor volume was measured by caliper twice weekly. Primary tumor volume was calculated according to NCl protocol (TV=1/2ab2, where a and b are long and short diameters of tumor size in mm, Teicher B. Anticancer drug development guide, Humana Press, 1997, Chapter 5, page 92). Calculation values were documented as mean and standard deviation.

Treatment of Animals

Tumor-bearing mice were randomized when the tumor volume was roughly 100 mm³ (n=10 for each group). Each group was closely matched before treatment, which began 20 days after tumor cell injection. Group A: Vehicle group—received 10 ml/kg PBS buffer intraperitoneally (i.p.) once weekly. Group B: trastuzumab was administered i.p. at a loading dose of 30 mg/kg, followed by once weekly doses of 15 mg/kg (maintenance dose). Group C: pertuzumab was administered i.p. at a loading dose of 30 mg/kg, followed by once weekly doses of 15 mg/kg (maintenance dose). Group D: Both antibodies, trastuzumab and pertuzumab, were given in the same dose regimen and time schedule as the monotherapies.

Evaluation of Metastasis

Spread of tumor cells into the lung was determined in sacrificed animals. Metastasis was measured according to Schneider, T., et al., Clin & Expt Metastasis 19 (2002). 571-582. Briefly, lung tissue was harvested and human Alu sequences were quantified by real-time PCR. Higher human DNA levels, quantified by real-time PCR, correspond to higher levels of metastasis.

Results

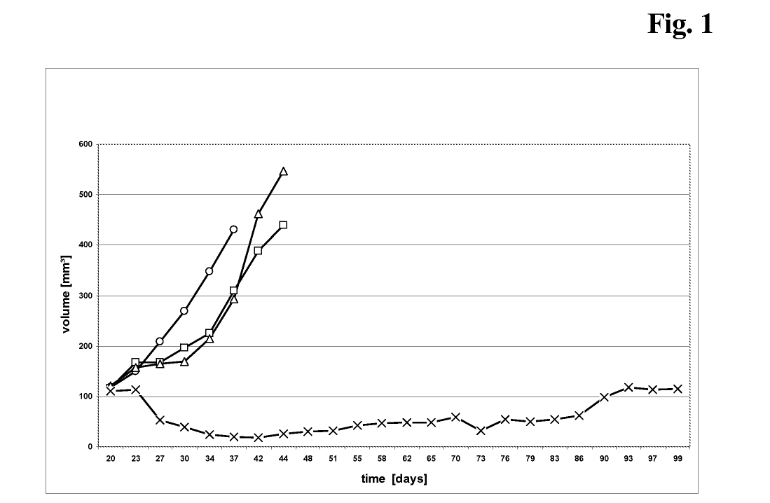

The effect of treatment on primary tumor growth is shown in FIG. 1 and Table 3. Primary tumors in the vehicle group (Group A) grew rapidly and mice were sacrificed 37 days after injection of tumor cells because of ulceration of tumors and the development of clinical symptoms. Treatment with trastuzumab (Group B) or pertuzumab (Group C) showed no significant influence on tumor growth and mice had to be sacrificed 44 days after tumor cell injection because mice were in bad clinical conditions. However, the combination treatment with trastuzumab and pertuzumab (Group D) resulted in significant inhibition of tumor growth and 6 of 10 animals had complete tumor regression. Compared to both mono therapies, the combination treatment resulted in an increase in life time (99 days compared to 44 days). Treatment was well tolerated in all animals of the combination group.

TABLE 3
Antitumor activity of a) trastuzumab, b) pertuzumab and c) combined trastuzumab
and pertuzumab treatment on tumor growth (data for FIG. 1). Mean tumor
volume in mm³ is reported and the standard deviation (SD).

| | | | | | | | Trastuzumab + | |
|---|---|---|---|---|---|---|---|---|
| Day | Vehicle (A) | SD | Trastuzumab (B) | SD | Pertuzumab (C) | SD | Pertuzumab (D) | SD |
| 20 | 118 | 31 | 117 | 36 | 120 | 31 | 110 | 33 |
| 23 | 150 | 30 | 167 | 40 | 157 | 57 | 113 | 44 |
| 27 | 209 | 51 | 168 | 71 | 164 | 77 | 53 | 23 |
| 30 | 269 | 76 | 196 | 79 | 169 | 82 | 39 | 24 |
| 34 | 348 | 114 | 226 | 134 | 214 | 114 | 24 | 25 |
| 37 | 431 | 138 | 310 | 169 | 293 | 162 | 19 | 25 |
| 42 | | | 388 | 188 | 462 | 275 | 17 | 23 |
| 44 | | | 440 | 226 | 547 | 315 | 26 | 36 |
| 48 | | | | | | | 30 | 42 |
| 51 | | | | | | | 32 | 48 |
| 55 | | | | | | | 42 | 71 |
| 58 | | | | | | | 46 | 81 |
| 62 | | | | | | | 49 | 94 |
| 65 | | | | | | | 49 | 92 |
| 70 | | | | | | | 59 | 123 |
| 73 | | | | | | | 31 | 41 |
| 76 | | | | | | | 54 | 67 |
| 79 | | | | | | | 51 | 66 |
| 83 | | | | | | | 55 | 70 |
| 86 | | | | | | | 63 | 90 |
| 90 | | | | | | | 98 | 129 |
| 93 | | | | | | | 118 | 150 |
| 97 | | | | | | | 113 | 141 |
| 99 | | | | | | | 114 | 152 |

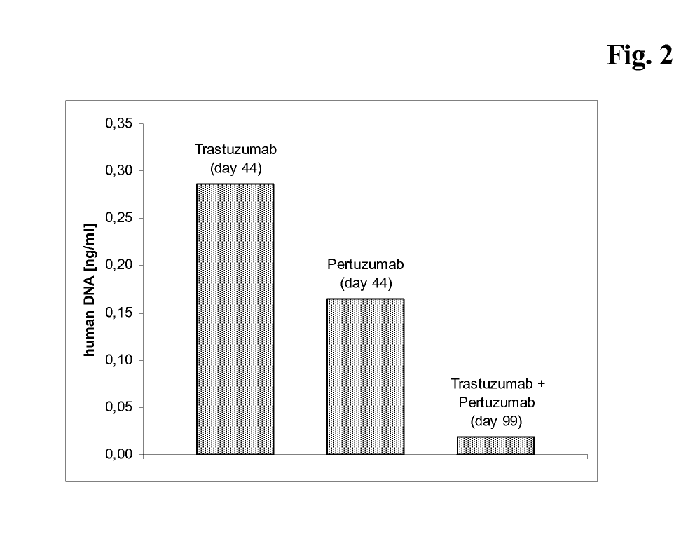

The effect of treatment on the prevention or reduction of lung metastasis is shown in FIG. 2 and table 4. The combination treatment of the primary tumor of trastuzumab and pertuzumab resulted in a sharp decrease of metastasis compared to monotherapy with trastuzumab or pertuzumab. Levels of human Alu sequences (correlating to invasion of tumor cells into secondary tissue) are significantly lower in animals treated with a combination therapy compared to animals treated with trastuzumab or pertuzumab alone. This surprising effect on metastasis is in contrast with the effect seen with cytotoxic drugs (Geldof et al., Anticancer Res 8 (1988) 1335-40; Murphy, J. Clin. Oncol. 11 (1993) 199-201; and De Larco et al., Cancer Res. 61 (2001) 2857-61).

TABLE 4
Effect of treatment on lung metastasis. Alu DNA was quantified
by real-time PCR and is reported for each animal.

| | | | Trastuzumab + |
|---|---|---|---|
| | Trastuzumab (B) | Pertuzumab (C) | Pertuzumab (D) |
| | (day 44) | (day 44) | (day 99) |
| human | 1.609 | 4.919 | 0.017 |
| DNA | 0.084 | 0.123 | 0.031 |
| [ng/ml] | 0.586 | 0.067 | 0.037 |
| | 0.055 | 1.110 | 0.024 |
| | 2.919 | 0.090 | 0.016 |
| | 0.078 | 0.515 | 0.040 |
| | 2.741 | 0.165 | 0.018 |
| | 0.017 | 0.060 | 0.018 |
| | 0.340 | 0.463 | 0.018 |
| | 0.232 | | |
| median | 0.29 | 0.16 | 0.02 |

1. A method for treating HER2 positive cancer or metastasis of HER2 positive cancer in a patient, who does not respond to a monotherapy with trastuzumab or to a monotherapy with pertuzumab or to successive monotherapies with trastuzumab and pertuzumab, comprising co-administering to said patient a therapeutically-effective amount of trastuzumab and pertuzumab simultaneously or sequentially.

2. A method according to claim 1 wherein said patient does not respond to a first-line monotherapy with trastuzumab.

3. A method according to claim 1 wherein said patient does not respond to monotherapy with trastuzumab or pertuzumab.

4. A method according to claim 1 wherein said method is for treating HER2 positive cancer in a patient.

5. A method according to claim 1 wherein said method is for treating metastasis of HER2 positive cancer in a patient and said patient does not respond to a monotherapy with trastuzumab or to a monotherapy with pertuzumab.

6. A method according to claim 1 wherein said co-administration of trastuzumab and pertuzumab comprises the administration of an initial dosage of said trastuzumab or said pertuzumab in an amount of from about 1 μg/kg to about 50 mg/kg.

7. A method according to claim 1 wherein said co-administration of trastuzumab and pertuzumab comprises the administration of an initial dosage of said trastuzumab or said pertuzumab in an amount of from about 0.05 mg/kg to about 10 mg/kg.

8. A method according to claim 1 wherein said HER2 positive cancer is a primary tumor.

9. A method according to claim 1 wherein said HER2 positive cancer is a locally advanced cancer or a metastatic cancer.

10. A method according to claim 1 wherein said HER2 positive cancer is selected from the group consisting of: breast cancer; lung cancer; colon cancer; and prostate cancer.

11. A method according to claim 1 wherein said HER2 positive cancer is breast cancer.

12. A method according to claim 1 wherein said metastasis is a metastasis of the lung.

13. A kit which comprises trastuzumab and pertuzumab and a package insert instructing the user to co-administer trastuzumab and pertuzumab to a patient suffering from HER2 positive cancer who does not respond to a monotherapy with trastuzumab or a monotherapy with pertuzumab.

14. A kit according to claim 12 wherein said patient does not respond to a first-line monotherapy with trastuzumab.
